(19) United States
(12) Patent Application Publication
(10) Pub. No.: US 2017/0333063 A1
(43) Pub. Date: Nov. 23, 2017

(54) Tongue cleaner with suction and irrigation ports.

(71) Applicants: Vinay Jivan, Dublin, CA (US); Paritosh Patel, Pismo Beach, CA (US); Anita Jivan, Dublin, CA (US)

(72) Inventors: Vinay Jivan, Dublin, CA (US); Paritosh Patel, Pismo Beach, CA (US); Anita Jivan, Dublin, CA (US)

(21) Appl. No.: 15/159,812

(22) Filed: May 20, 2016

(51) Int. Cl. A61B 17/24

(52) U.S. Cl. CPC A61B 17/244; A61B 2217/005; A61B 2217/007

(57) ABSTRACT

A tongue cleaner that can be used by an operator or an individual that is capable of being connected to an apparatus that has suction/vacuum and a fluid reservoir system. The head part of the tongue cleaner includes a scraper edge, irrigation ports and a cavity well housed with suctions ports. The handle part of the cleaner includes the conduits for carrying the egress and ingress fluids to the head, a connection ports for the tubing that connects to the apparatus and a valve system that allows the user to control the irrigation and suction. The head part of the tongue cleaner is made to be disposable so that every new user would be using a fresh sterilized cleaner.

### FIELD OF THE INVENTION

This invention relates to a system that helps maintain good oral hygiene by cleaning the surface of your tongue. Particularly, this invention relates to a tongue scraper that safely removes plaque, bacteria and debris buildup from tongue surfaces better than brushing alone.

### BACKGROUND OF THE INVENTION

A toothbrush is used to clean teeth, but only a few people use the conventional brush to brush their tongue to remove plaque and debris from their tongue. Conventional toothbrushes with bristles are often uncomfortable to use on the tongue and hence many people don't use it to clean their tongue. In Asian countries people have been using tongue scrapers for many centuries. These scrapers are typically a U shape curved metal, made from stainless steel or copper, and are used to clean the tongues.

These scrapers help remove plaque and debris from the tongue but do a poor job in killing the bacteria that resides on your tongue. Hence, there is an unmet need that toothbrushes and tongue scrapers need to overcome in order to enhance oral hygiene.

### SUMMARY OF THE INVENTION

This invention centers on a unique design of a tongue scraper that can deliver cleaning, disinfecting, or other solutions and at the same time allowing for optimum removal of plaque and debris from the tongue.

The present disclosure provides an oral care implementation that includes a disposable head that forms the scraper edge, which channels the plaque and debris to the suction ports within the head so that they can be removed effectively. Irrigation ports are also located in the head to provide rinsing, anti-bacterial medication and other fluids to be applied to the tongue during the cleaning process. The head is designed to be disposable and replaceable and it connects to the handle part of the design.

One end of the handle is used to attach the head and the other end to apparatus that provides the vacuum and rising, anti-bacterial fluids. Built within the handle is a control valve that the user can use to control the amount of suction and irrigation.

### BRIEF DESCRIPTION OF DRAWINGS

FIG. 1: Is a side view of the tongue cleaner in accordance with and/or use with the present invention.

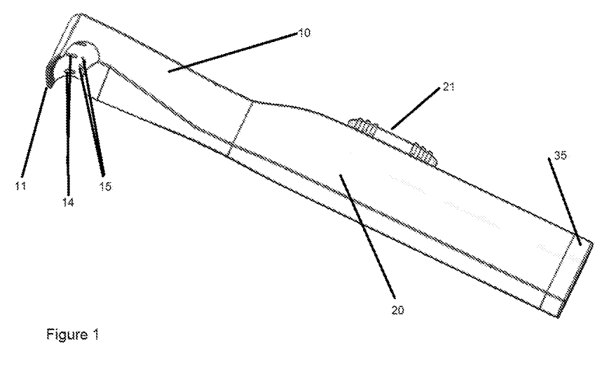

FIG. 2: Shows a close up image of the head and the parts that make up the head. The scraper edge is beveled to ensure that it is smooth yet thin enough to scrape the tongue without any injuries. It also shows the location of the suction holes, the cavity well which aids in the storage and removal of the debris with respect to the irrigation holes, which are used to spray the fluids such as the disinfecting solution.

FIG. 3: Show the fluid conduits that are built into the handle part of the design and the flow control button.

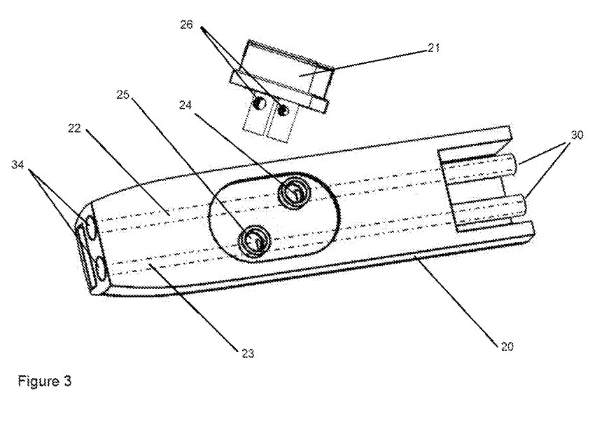

FIG. 4: Shows a cross section view of the fluid conduits inside the head.

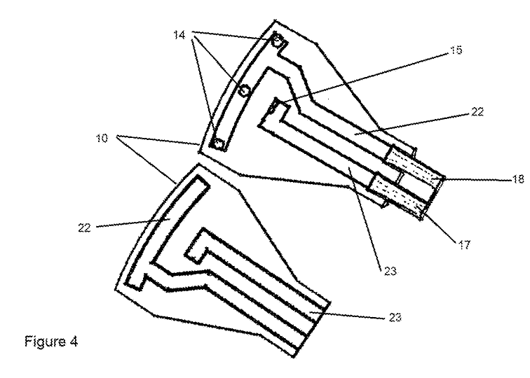

FIG. 5: Shows the end of the handle, the ports that are used to connect to the suction/vacuum and the fluid delivering apparatus.

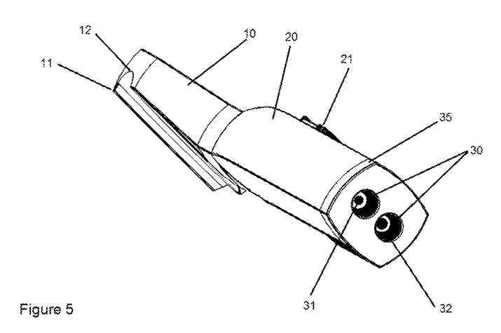

### REFERENCE NUMERALS IN DRAWINGS

10 Disposable head part of cleaner

11 Beveled scraper edge

12 Cavity wall

14 Suction holes located in cavity wall of head

15 Irrigation holes located in head

16 Insert key

17 Irrigation connector

18 Suction connector

20 Handle part of scraper

21 Oval flow control button

22 Built in conduits for suction

23 Build in conduits for irrigation

24 Blocking plunger hole controls suction

25 Blocking plunger hole controls irrigation

26 Flow control ports

30 Connection ports to apparatus

31 Inlet/Ingress port to cleaner

32 Outlet/Egress port to cleaner

33 Connection keyed to ensure correct connection.

34 Connection port Lo cleaning head

35 Strain relief for tubing

### DETAILS OF THE INVENTION

The present invention provides a tongue cleaner designed to aid in a wide variety of oral hygiene. The low profile head (10) incorporates:—a beveled scraper edge (11), suction ports (14) for vacuum suction, irrigation ports (15) for installation of disinfecting/rinsing fluid, a cavity well (12) that acts as a for temporary holding for the debris that is being removed. The cavity well (12) and its shape can also aid in the cleaning. For example, a cylindrical shape cavity with suction ports (14) at the top creates a swirl of unwanted fluids and debris as the tongue cleaner is stroked. With the location of the suction ports (14) at the top it ensures effective removal of the debris without the tongue being stuck to the tongue scraper (11) due to the suction.

The irrigation ports (15) would normally be located directly opposite from the blade edge (11) allowing the rinsing agent or other fluids to be directed as closely to the tongue surface that is about to be scraped. This directed irrigation would aid in releasing unwanted plaque and debris while also disinfecting the tongue during the scraping process.

In this design the tongue cleaner handle (20) has the fluid channels built into it that connect to the tongue cleaner head (10) and to the apparatus (30) that would provide the vacuum suction and pressurized irrigation/disinfecting fluid.

The connection port (34) at the head end of the handle can be made to have a keyed (33) quick connector allowing for faster connection/disconnection of the head (10). This would allow for dentists, oral hygienists and other individuals to quickly change and dispose of the used head (10) for a new one. to the tubing that connects the tongue cleaner to the apparatus that provides the vacuum and pressurized fluid.

Located in the handle can be an oval shape button (21) that can be used to selectively control the suction, irrigation or both depending as to where the button is pushed. For example pushing the oval button (21) on the top would control the irrigation, the bottom the suction and in the middle both the irrigation and suction. Scrapping, irrigation and suction as described can hence all be control by the user through the handle without any cumbersome interaction. The control buttons don't necessary have to be mechanical valves, the buttons could be a pressure switches that could be connected wired or wireless to the irrigation and suction/vacuum apparatus directly to provide control to the user.

The scraper edge (11) can be plastic, metal or even rubber and typically the edge would have to be beveled.

All three actions cleaning, irrigating/disinfecting and removal of debris can be provided by the tongue cleaner. This invention as it stands allows for easier insertion into ones mouth without fear and has additional psychological and social benefits from that of the traditional U-shape metal tongue cleaner. Plus traditional tongue cleaners don't irrigate/disinfect the tongue.

The handle (20) with the built in fluid conduits (22), (23), the connection ports (30) and the in house go button (21) to control the suction and irrigation further reduce clutter and provide ease of use to the user.

1. A tongue cleaner for use by an operator or individual that is capable to be connected to an apparatus that can provide suction/vacuum and also be able to deliver fluids under slight pressure. The tongue cleaner comprising:
A head (10) that has a beveled edge, which can be made from plastic metal or rubber that forms the scraper (11), a cavity well (12) that acts as a temporary storage for the debris, suction ports (14) positioned with the cavity well (12), irrigation ports (15) that would normally be directly opposite from the blade edge (11) allowing the rinsing agent or other fluids to be directed as closely to the tongue surface that is about to be scraped.
A handle (20) that has built in conduits to transfer the fluids to and from the head (10) to the ingress and egress ports (30) that connects to the suction and fluid delivery apparatus. The ingress egress ports could have a quick keyed (33) disconnect feature that would allow one to quickly change, and dispose of used head (10) devices.

2. The tongue cleaner of claim 1, control of the suction and irrigation ports is made possible by having a control button (21) built in the handle to accommodate single handed operation of the tongue cleaner.

3. The tongue cleaner of claim 1, position of the suction ports (14) is optimized to remove the debris and waste fluids without interfering with the tongue.

4. The tongue cleaner of claim 1, the head (10) is configured to be removable from the handle (20) and hence different size heads (10) can be supported.

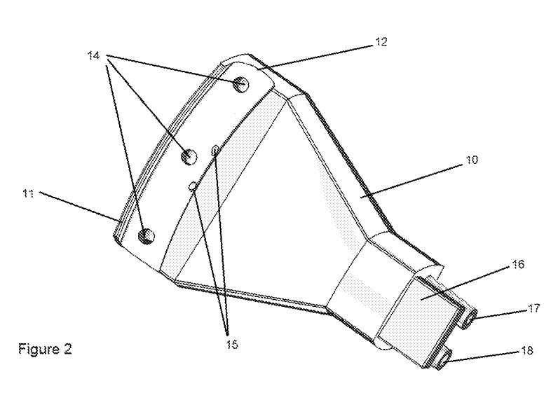

5. FIG. 2 shows an example of the tongue cleaner claim 1. Other possible shapes for a tongue cleaner with a head (10) having irrigation ports (15), cavity wells (12) and suction ports (14) all in one and are part of this claim.
